(19) United States
(12) Patent Application Publication
(10) Pub. No.: US 2009/0142391 A1
(43) Pub. Date: Jun. 4, 2009

(54) Conjugated RNAi Therapeutics

(76) Inventor: Mitchell W. Mutz, La Jolla, CA (US)

Correspondence Address: MINTZ, LEVIN, COHN, FERRIS, GLOVSKY AND POPEO, P.C, 5 Palo Alto Square - 6th Floor, 3000 El Camino Real, PALO ALTO, CA, 94306-2155, US

(21) Appl. No.: 12/191,940

(22) Filed: Aug. 14, 2008

Related U.S. Application Data
(60) Provisional application No. 60/964,748, filed on Aug. 14, 2007.

(51) Int. Cl. A61K 9/127; A61K 31/7052; A61P 43/00

(52) U.S. Cl. 424/450; 514/44

(57) ABSTRACT

A method for modulating at least one pharmacokinetic property of a drug which degrades mRNA upon administration to a host by an siRNA mechanism is provided. In a further embodiment of this invention, a bifunctional compound comprising an siRNA and a recruiter moiety are provided. The recruiter moiety may be lipophilic and may enable the siRNA to cross cell membranes and then targets an endogenous, intracellular protein to allow better distribution of the therapeutic into the cell and therefore, higher efficacy.

### CROSS-REFERENCE TO RELATED APPLICATIONS

This application claims priority to U.S. Provisional Patent Application No. 60/964,748, filed Aug. 14, 2007, which is incorporated by reference herein in its entirety.

### TECHNICAL FIELD

This invention relates generally to pharmacology and more specifically to the modification of active agents to give them more desirable properties.

### BACKGROUND

RNA interference or RNAi is a promising new approach towards making therapeutics which have more specificity and lower toxicity. RNAi is exquisitely selective for specific targets since it is directed towards gene-specific transcripts. Moreover, unlike anti-sense strategies, RNAi is a catalytic process where the same RNAi can be used many times to effect cleavage of many mRNA molecules. In humans, one molecule of siRNA can cause the cleavage of 60-70 mRNA molecules. For this reason, RNAi has attracted great interest as new method for the treatment of disease caused by the expression of a disease-causing protein. RNAi can be achieved via the use of a range of approaches: chemically synthesized small interfering RNA (siRNA) or endogenous expression of microRNA (miRNA), siRNA, or small hairpin RNA (shRNA). Despite the promise of this technology, there are technical hurdles to overcome: delivery of siRNA to cells is typically not sufficient to allow a therapeutic concentration of siRNA for gene silencing. In addition delivering too much siRNA can activate immune responses in a concentration-dependent manner, and this leads to non-specific gene silencing. Among the current approaches to solving the exogenous, systemic siRNA delivery problem are based on chemically modified siRNA, lipid encapsulation, polymeric carriers, and bioconjugation to biopolymers, but none have emerged as an optimal, general solution for the delivery of siRNA.

An emerging technology for the intracellular delivery and enhanced stability of siRNAs is the use of lipid conjugation. Enhanced lipophilicity is known to increase the membrane permeability of therapeutics and generally enhances the systemic exposure of a drug by increasing the elimination half-life. Additionally, enhancing the binding affinity of siRNA for endogenous human serum albumin by the use of cholesterol-siRNA conjugates, has also been shown to increase the elimination half life of siRNAs from 6 minutes to 95 minutes (Soutschek, J, et al. Nature, vol. 32, pp 173-178) by the introduction of cholesterol to the 3′ end of siRNAs. One disadvantage of the strategy for introducing conjugates to siRNA which enhance albumin binding is the tendency to enhance extra-cellular concentration of siRNA, as opposed to promoting intracellular sequestration. An approach to enhance lipophilicity and enhance intracellular distribution of siRNAs is of therapeutic interest. Moreover, increasing the elimination half life of siRNA is important to reduce the risk of side effects and lower the cost of administration of therapeutic siRNA. Lastly, the introduction of conjugates with independent biological activity such as cholesterol runs the risk that they may have their own side effects. Thus, the use of biologically silent conjugates is of interest to avoid additional side effects.

### SUMMARY OF THE INVENTION

In an embodiment of this invention, a method for modulating at least one pharmacokinetic property of a drug which degrades mRNA upon administration to a host by an siRNA mechanism is provided. In a further embodiment of this invention, a bifunctional compound comprising an siRNA and a recruiter moiety are provided. The recruiter moiety is lipophilic and enables the siRNA to cross cell membranes and then targets an endogenous, intracellular protein to allow better distribution of the therapeutic into the cell and therefore, higher efficacy. The recruiter moiety is preferably biologically silent and does not have toxic side effects independent of the siRNA at medically relevant dosages.

In a further aspect of the invention, biasing the drug to remain inside cells increases efficacy by a two-fold mechanism: avoiding extracellular RNAse enzymes and avoiding intracellular degradation by enzymes via an association with a non-target intracellular protein which confers protection from intracellular enzymes. The non-target protein must still allow interaction of the siRNA to the RNA-induced silencing complex (RISC) and optimally enhances the binding affinity for RISC, measured directly by the association constant, Kₐ, or indirectly by IC₅₀ measurements. The intracellular bias is created using ligands for intracellular proteins and ligands which avidly target intracellular proteins. In an additional aspect of the invention, the bifunctional siRNA drug has lower toxicity than the parent compound because a lower dose is required to achieve equivalent efficacy due to enhanced concentration/hour (area under the curve) and lower elimination half life.

In another embodiment of the invention, the recruiter moiety binds to the passenger (antisense) strand of the siRNA, allowing the guide strand to remain in the RISC complex.

### FIGURES

FIG. 1 depicts the structure of SLF linked to a modular linker and siRNA. Due to the modular nature of the synthesis, the linker group and siRNA may be readily altered.

In FIG. 2, the left side depicts the bimodal binding character of FK506 whereby it binds both FKBP and calcineurin. The schematic on the right depicts how the calcineurin-binding mode can be eliminated by substituting a linker and target binding moiety. In this manner, FK506 can simultaneously target FKBP and bind a second protein. Synthetic ligands with no affinity for calcineurin such as SLF may also be used.

In FIG. 3, the left side illustrates sample linkers that could be employed in a modular synthetic scheme.

FIG. 4 exhibits a synthetic scheme to make an SLF-maleimide derivative to conjugate to a thio modified siRNA.

FIG. 5 illustrates the efficacy of a bifunctional paclitaxel drug in cell culture.

FIG. 6 shows the difference in partitioning between the extra- and intracellular space due to the presence of the recruiter moiety in an in vivo mouse model study.

FIG. 7 shows the effect of area under the curve (AUC) for a bifunctional compound in mice vs. a monofunctional compound. Compound was administered via a tail vein injection to mimic intravenous drug administration. Enhanced lipophilicity combined with the high bioavailability of an intracellular target may account for the increased AUC.

FIG. 8 shows the efficacy of the paclitaxel bifunctional in a xenograft tumor mouse model vs a vehicle control containing the Cremaphor-ethanol solvent only.

FIGS. 9-11 show experimental data obtained as described in Example 6.

### DETAILED DESCRIPTION OF PREFERRED EMBODIMENTS

Before describing the present invention in detail, it is to be understood that this invention is not limited to specific solvents, materials, or device structures, as such may vary. It is also to be understood that the terminology used herein is for the purpose of describing particular embodiments only, and is not intended to be limiting.

As used in this specification and the appended claims, the singular forms “a,” “an,” and “the” include both singular and plural referents unless the context clearly dictates otherwise. Thus, for example, reference to “an active ingredient” includes a plurality of active ingredients as well as a single active ingredient, reference to “a temperature” includes a plurality of temperatures as well as single temperature, and the like.

The term “bifunctional compound” refers to a non-naturally occurring compound that includes a recruiter moiety and a drug moiety, where these two components may be covalently bonded to each other either directly or through a linking group. The term “drug” refers to any active agent that affects any biological process including RNA molecules involved in an RNAi process. Bifunctional compounds may have more than two functionalities.

The term RNAi therapeutic includes any RNA molecule which is used to modulate the level of an mRNA transcript in a cell. Such therapeutics include, but are not limited to, shRNA, miRNA, and siRNA.

The recruiter moiety may be a peptide or protein and may also be an enzyme or nucleic acid or other biomoiety and is covalently attached to the RNAi therapeutic.

By “pharmacologic activity” is meant an activity that modulates or alters a biological process so as to result in a phenotypic change, e.g. cell death, cell proliferation etc.

By “pharmacokinetic property” is meant a parameter that describes the disposition of an active agent in an organism or host. Representative pharmacokinetic properties include: drug half-life, hepatic first-pass metabolism, volume of distribution, degree of blood serum protein, e.g. albumin, binding, etc, degree of tissue targeting, cell type targeting.

By “half-life” is meant the time for one-half of an administered drug to be eliminated through biological processes, e.g. metabolism, excretion, etc.

By “hepatic first-pass metabolism” is meant the propensity of a drug to be metabolized upon first contact with the liver, i.e. during its first pass through the liver.

By “volume of distribution” is meant the distribution and degree of retention of a drug throughout the various compartments of an organism, e.g. intracellular and extracellular spaces, tissues and organs, etc.

The term “efficacy” refers to the effectiveness of a particular active agent for its intended purpose, i.e. the ability of a given active agent to cause its desired pharmacologic effect.

The term “host” refers to any mammal or mammalian cell culture or any bacterial culture.

Where the term cancer is used, it is understood that the invention may be employed on relative chemotherapeutics such as found in other any type of cancer including those cancers found in non-human species or human variants.

Where the term “intracellular” protein is used, this includes any protein that resides predominantly in the intracellular space but may optionally reside as a transmembrane or receptor protein.

The term “biomoiety” refers to a protein, DNA, RNA, ligand, carbohydrate, lipid, or any other component molecule of a prokaryotic or eukaryotic organism.

The term “non-pharmacokinetic properties” may include binding constants, off rate or on rate constants of the RNAi therapeutic moiety and recruiter for their targets. Additional properties may include drug solubility, formulation, and permeability across membranes.

The term “exon skipping” refers to manipulation of pre-mRNA splicing to produce new forms of the mRNA transcript. This technique has therapeutic benefit where the new mRNA transcript produces a protein with enhanced therapeutic benefit or lower toxicity than the “parent” or unmanipulated pre-mRNA moiety. Multiple exons may be removed, as in the simultaneous removal of exons 6 and 8 for the treatment of muscular dystrophy (McClorey, G., et al. Gene Therapy, 13, 1373-1381, 2006). Exon skipping may be seen as a distinct application from gene silencing or modulating the expression of a gene without changing the reading frame or adding or deleting transcribed segments. Exon skipping has been described in Mann, C. J., et al. Proc. Natl. Acad. Sci., 97, 13714-13719 (2000).

Where FK506 is used, variants or analogs of FK506 are included, such as rapamycin, pimecrolimus, or synthetic ligands of FK506 binding proteins (SLFs) such as those disclosed in U.S. Pat. Nos. 5,665,774, 5,622,970, 5,516,797, 5,614,547, and 5,403,833 or described by Holt et al., “Structure-Activity Studies of Synthetic FKBP Ligands as Peptidyl-Prolyl Isomerase Inhibitors,” Bioorganic and Medicinal Chemistry Letters, 4(2):315-320 (1994).

The term “siRNA” may refer to any form of RNA (double stranded RNA, microRNA, or short hairpin RNA) which may be used to silence a gene or create an alternative form of a transcript or protein by a different mechanism such as exon skipping.

In an embodiment of this invention, a method for modulating at least one pharmacokinetic property of an siRNA upon administration to a host is provided. One administers to the host an effective amount of a bifunctional compound of less than about 50000 Daltons comprising the siRNA therapeutic or an active derivative thereof and a recruiter moiety. The recruiter moiety binds to at least one intracellular protein. The bifunctional compound has at least one modulated pharmacokinetic property upon administration to the host as compared to a free drug control that comprises the RNAi therapeutic.

Bifunctional compounds in general have aroused considerable interest in recent years. See, for example, U.S. Pat. Nos. 6,270,957, 6,316,405, 6,372,712, 6,887,842, 6,921,531 and PCT publication WO2007/53792. ConjuChem (Montreal, Canada) scientists have shown that covalent coupling of insulin to human serum albumin can improve the half-life from 8 hours to over 48 hours. Xenoport (Santa Clara, Calif.) has pioneered attachment of receptor ligands to improve drug uptake and distribution. Similarly, siRNA-lipid conjugates have shown therapeutic promise as anti-leukemic drugs. Human trials of methotrexate-albumin conjugates revealed that the modified methotrexate had half-lives of up to two weeks compared with 6 hours for unmodified methotrexate. Other examples include PEGylation of growth factors and attachment of folate groups that “target” anti-cancer drugs. All these strategies use modification of a “parent” drug to provide new binding profiles or enhanced protection from degradation.

More recently, a team attached SLF to ligands for amyloid beta. Amyloid beta oligomers are believed to underlie the neuropathology of Alzheimer's disease. Therefore, methods to decrease amyloid aggregation are of therapeutic interest. Amyloid ligands, such as congo red or curcumin, can be synthetically coupled to FK506 or SLF. The resulting bifunctional compound binds both FKBP and amyloid beta. These molecules are potent inhibitors of amyloid aggregation and they block neurotoxicity in cell culture. Moreover, these ligands penetrate the blood-brain barrier and may assist biodistribution of siRNA to the brain. See Jason E. Gestwicki et al., “Harnessing Chaperones to Generate Small-Molecule Inhibitors of Amyloid β Aggregation,” Science 306:865-69 (2004).

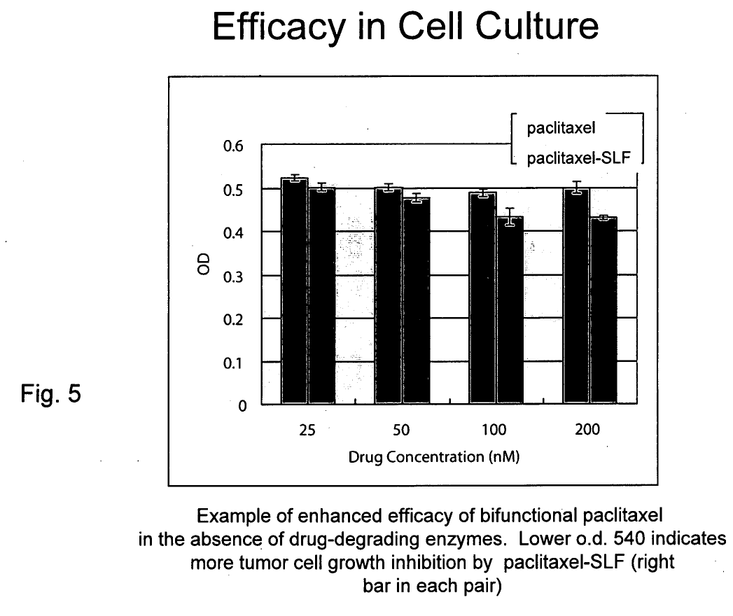

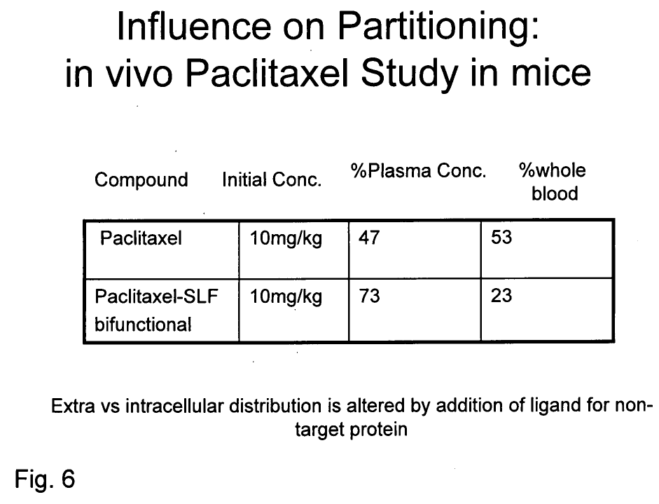

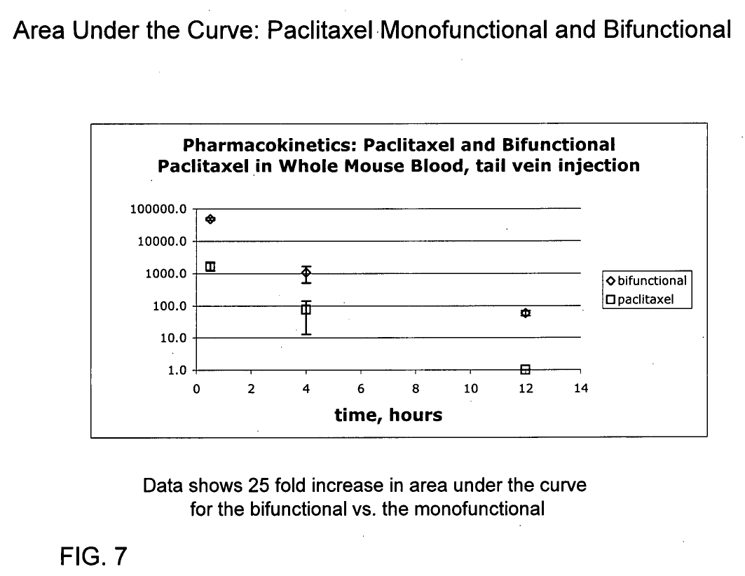

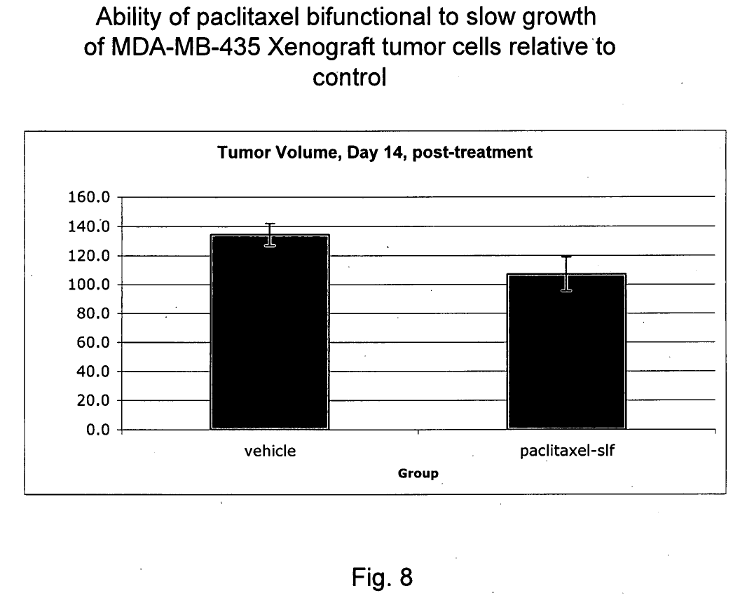

The type of improvements achievable with bifunctional molecules are illustrated in FIGS. 5-8, which show results from a paclitaxel-SLF conjugate.

Bifunctional compounds of the type employed in the present invention are generally described by the formula:

X-L-Z

wherein:

X is a drug moiety, for example an RNAi therapeutic;

L is a bond or linking group; and

Z is a recruiter moiety,

Thus, as may be seen, a bifunctional compound is a non-naturally occurring or synthetic compound that is a conjugate of a drug or derivative thereof and a recruiter moiety, where these two moieties are optionally joined by a linking group.

In bifunctional compounds used in the invention, the recruiter and drug moieties may be different, such that the bifunctional compound may be viewed as a heterodimeric compound produced by the joining of two different moieties. In many embodiments, the recruiter moiety and the drug moiety are chosen such that the corresponding siRNA target and any binding partner of the recruiter moiety, e.g., a recruiter protein to which the recruiter moiety binds, do not naturally associate with each other to produce a biological effect.

The mass of an RNAi therapeutic is typically at least 4000 daltons. As such, the molecular weight of the bifunctional compound is generally at least about 4000 D, usually at least about 6000 D and more usually at least about 10000 D. The molecular weight may be less than about 8000 D, about 12000 D, about 15000 D, or about 20000 D, and may be as great as 30000 D or greater, but usually does not exceed about 50000 D. The preference for small molecules is based in part on the desire to facilitate oral administration of the bifunctional compound. Molecules that are orally administrable tend to be small.

The recruiter moiety modulates a pharmacokinetic property, e.g. half-life, hepatic first-pass metabolism, volume of distribution, degree of albumin binding, and intra- vs. extracellular distribution upon administration to a host as compared to free drug control. By modulated pharmacokinetic property is meant that the bifunctional compound exhibits a change with respect to at least one pharmacokinetic property as compared to a free drug control. For example, a bifunctional compound of the subject invention may exhibit a modulated, e.g. longer, half-life than its corresponding free drug control. Similarly, a bifunctional compound may exhibit a reduced propensity to be eliminated or metabolized upon its first pass through the liver as compared to a free drug control. Likewise, a given bifunctional compound may exhibit a different volume of distribution that its corresponding free drug control, e.g. a higher amount of the bifunctional compound may be found in the intracellular space as compared to a corresponding free drug control. Analogously, a given bifunctional compound may exhibit a modulated degree of albumin binding such that the drug moiety's activity is not as reduced, if at all, upon binding to albumin as compared to its corresponding free drug control. In evaluating whether a given bifunctional compound has at least one modulated pharmacokinetic property, as described above, the pharmacokinetic parameter of interest is typically assessed at a time at least 1 week, usually at least 3 days and more usually at least 1 day following administration, but preferably within about 6 hours and more preferably within about 1 hour following administration.

The linker L, if not simply a bond, may be any of a variety of moieties chosen so that they do not have an adverse effect on the desired operation of the two functionalities of the molecule and also chosen to have an appropriate length and flexibility. The linker may, for example, have the form F₁—(CH₂)ₙ—F₂ where F₁ and F₂ are suitable functionalities. A linker of this sort comprising an alkylene group of sufficient length may allow, for example, for the free rotation of the drug moiety even when the recruiter moiety is bound. Alternatively, a stiffer linker with less free rotation may be desired. The hydrophobicity or hydrophobicity of the linker is also a relevant consideration. FIG. 3 depicts some precursors which may be used for the linker (with the carboxyl functionality protected).

The drug moiety X may, in certain embodiments of the invention, preferably be an siRNA therapeutic. The drug moiety may also be in the form of short hairpin RNA (shRNA) or micro RNA (miRNA), which modulates the level of a therapeutically important mRNA or protein. The siRNA moiety preferably has a functionality which may readily and controllably be made to react with a linker precursor. The known siRNAs are subject to enzymatic degradation and clearance.

In general, the recruiter moiety Z will be one which is capable of reversible attachment to a common protein, meaning one which is abundant in the body or in particular compartments of the body or particular tissue types. Common proteins include, for example, FK506 binding proteins, cyclophilin, tubulin, actin, heat shock proteins, and albumin. Common proteins are present in concentrations of at least 10 nanomolar, preferably at least 100 nanomolar, more preferably at least 100 micromolar, and even more preferably 1 millimolar in the body or in particular compartments or tissue types. The recruiter moiety should, like the drug, have a moiety which is capable of reacting with suitable linkers.

It is desirable for at least some embodiments of the present invention that the binding of the recruiter moiety Z to a common protein be such as to sterically hinder the activity of common metabolic enzymes such as RNAse enzymes when the bifunctional compound is so bound. Persons of skill in the art will recognize that the effectiveness of this steric hindrance depends, among other factors, on the conformation of the common protein in the vicinity of the recruiter moiety's binding site on the protein, as well as on the size and flexibility of the linker. The choice of a suitable linker and recruiter moiety may be made empirically or it may be made by means of molecular modeling of some sort if an adequate model of the interaction of candidate recruiter moieties with the corresponding common proteins exists. The linker choice is critical since it must balance parameters of length, hydrophobicity, attachment point to the drug target, and attachment point to the ligand.

The attachment point and linker characteristics are preferably selected based on structural information such that the inhibitory potency of the RNAi therapeutic is preserved, giving the desired superior pharmacokinetic characteristics.

Where the recruiter moiety operates by binding a protein, it may be referred to as a “presenter protein ligand” and the protein which it binds to may be referred to as a “presenter protein.”

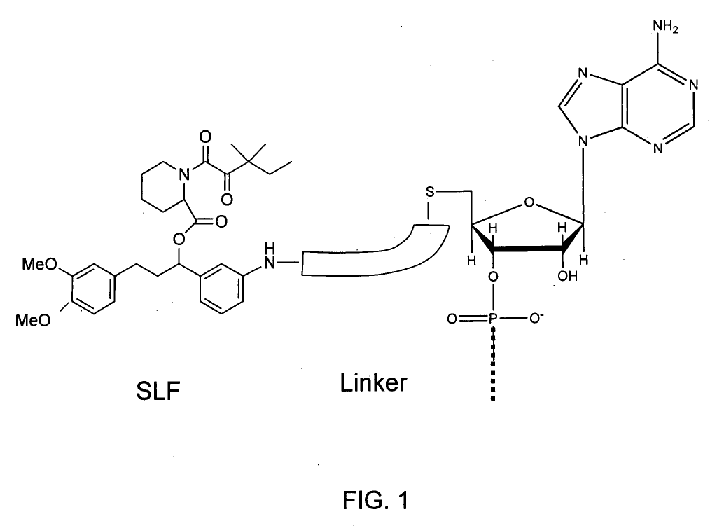

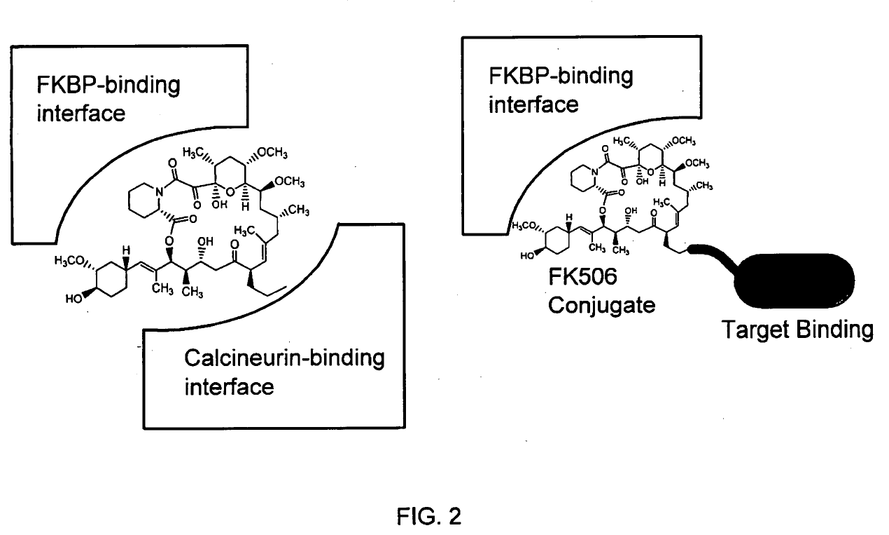

The recruiter moiety may be, for example, a derivative of FK506 (such as SLF) which has high affinity for the FK506-binding protein (FKBP), as depicted for example in FIG. 1. There are many synthetic ligands for FKBP. The abundance of FKBP (μmolar) in blood compartments, such as red blood cells and lymphocytes, makes it likely that a significant proportion of a dose of bifunctional compounds comprising FK506 would partition into blood cells, lymphocytes, and macrophages. A mechanism that tends to increase the portion of the chemotherapeutic dose that winds up in red blood cells and CD4+ lymphocytes will have a favorable effect on anti-cancer activity, as these sites are prime targets of chemotherapy. The steric bulk conferred by FKBP would hinder an RNAi therapeutic moiety from fitting into the binding pocket of intracellular enzymes (RNases) and so would prevent degradation via this class of enzymes.

An inactive form of FK506 may be preferable in many applications to avoid the possibility of side effects due to the possible interaction of the active FK506-FKBP complex with calcineurin. It may be advantageous to use FKBP binding molecules such as synthetic ligands for FKBP (SLFs) described by Holt et al., supra. This class of molecule is lower molecular weight than FK506, and that is generally advantageous for drug delivery and pharmacokinetics. For illustrative purposes, some diagrams will show examples of the use of FK506, though it should be understood that the same strategy can apply to other ligands of peptidyl prolyl isomerases such as the FKBP proteins and that ligands for other presenter proteins may be employed.

The value of FK506 and other FKBP binding moieties as recruiter moieties of the invention is further supported by the following. FK506 (tacrolimus) is an FDA-approved immunosuppressant. It has been determined that FK506 can be readily modified such that it loses all immunomodulatory activity but retains high affinity for FKBP. FKBP is an abundant chaperone that is particularly prevalent (˜μmolar) in red blood cells (rbcs) and lymphocytes. The complex between FK506-FKBP gains affinity for calcineurin and inactivation of calcineurin blocks lymphocyte activation and causes immunosuppression.

This interesting mechanism of action is believed to be a consequence of FK506's chemical structure. FK506 is bifunctional; it has two non-overlapping protein-binding faces. One side binds FKBP, while the other binds calcineurin. This property provides FK506 with remarkable specificity and potency. Moreover, FK506 has a long half-life in non-transplant patients (21 hrs) and excellent pharmacological profile. In part, this is because FK506 is unavailable to metabolic enzymes via its high affinity for FKBP, which favors distribution into protected cellular compartments (72-98% in the blood). It can be expected that suitable bifunctional compounds with an FKBP-binding recruiter moiety will likewise possess some favorable characteristics of inactive FK506, namely, good pharmacokinetics and blood cell distribution, membrane permeability, and long elimination half-life.

In general, the recruiter moiety will have a molecular weight less than about 2000 D, less than about 1800 D, less than about 1500 D, less than about 1100 D, or less than about 900 D or less than 500 daltons.

It is also possible to co administer the common protein to which the recruiter moiety binds with the bifunctional compound in order to modify the pharmacokinetics to a greater degree than would be possible with just the native concentration of the common protein.

In a further embodiment of this invention, a method is provided for synthesizing a bifunctional compound comprising an RNAi therapeutic functionality and the ability to bind to a common protein.

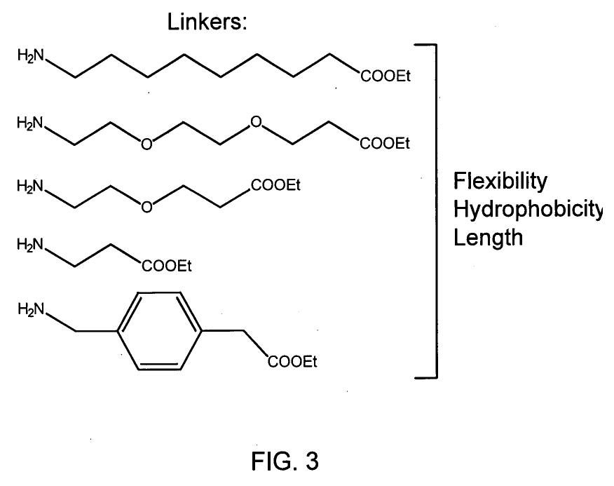

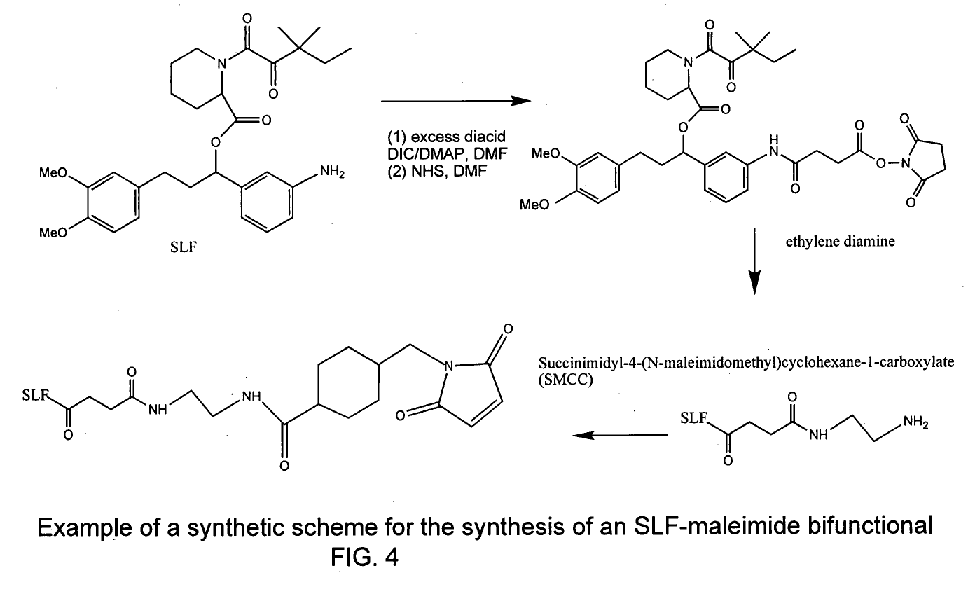

The synthesis of the bifunctional compound starts with a choice of suitable recruiter and drug moieties. It is desirable to identify on each of these moieties a suitable attachment point which will not result in a loss of biological function for either one. This is preferably done based on the existing knowledge of what modifications result or do not result in a biological function. On that basis, it may reliably be conjectured that certain attachment points on the pharmacokinetic and RNAi moieties do not affect biological function. Likewise, in FIG. 4, one sees a primary amine function on SLF, which, after modification to a maleimide, can serve as an attachment point to an introduced 5′ thiol moiety on siRNAs (Muratovska, A. et al. FEBS Letter, vol. 558, 2004, pp 63-68).

In a further aspect of the invention, a bifunctional compound comprising a RNAi therapeutic moiety with antiviral activity is formulated, for example in the form of a tablet, capsule, parenteral formulation, to make a pharmaceutical preparation. The pharmaceutical preparation may be employed in a method of treating a patient having cancer against which the RNAi therapeutic moiety is effective. For example, if the RNAi therapeutic moiety is effective against breast cancer, the pharmaceutical preparation may be administered to a patient suffering from breast cancer.

For the preparation of a pharmaceutical formulation containing bifunctional compounds as described in this application, a number of well known techniques may be employed as described for example in Remington: The Science and Practice of Pharmacy, Nineteenth Ed. (Easton, Pa.: Mack Publishing Company, 1995).

In a further embodiment, the invention relates to a method for treating a subject having a disease or at risk of developing a disease caused by the expression of a target gene. In this embodiment, bifunctional molecules comprising siRNA moieties can act as novel therapeutic agents for controlling one or more of cellular proliferative and/or differentiative disorders, disorders associated with bone metabolism, immune disorders, hematopoietic disorders, cardiovascular disorders, liver disorders, viral diseases, or metabolic disorders. The method includes administering a pharmaceutical composition of the invention to the patient (e.g., a human), such that expression of the target gene is silenced. Because of their high efficiency and specificity, the bifunctional molecules of the present invention may specifically target mRNA of target genes of diseased cells and tissues, as described below, at low dosages.

Examples of genes which can be targeted for treatment by the siRNA moiety include, without limitation, an oncogene, a cytokine gene, an idiotype (Id) protein gene, or a prion gene. The targeted gene may result in the expression of molecules that induce angiogenesis, adhesion molecules, or cell surface receptors. The targeted genes may pertain to proteins that are involved in metastasizing and/or invasive processes, or may be genes of proteases, genes of molecules that regulate apoptosis and the cell cycle. One may target, for example, the drug resistance 1 gene, NMDR1.

In the prevention of disease, the target gene may be one which is required for initiation or maintenance of the disease, or which has been identified as being associated with a higher risk of contracting the disease. In the treatment of disease, the siRNA moiety can be brought into contact with the cells or tissue exhibiting the disease. For example, an siRNA moiety substantially identical to all or part of a mutated gene associated with cancer, or one expressed at high levels in tumor cells, may be brought into contact with or introduced into a cancerous cell or tumor gene.

Examples of cellular proliferative and/or differentiative disorders include cancer, e.g., a carcinoma, sarcoma, metastatic disorder or hematopoietic neoplastic disorder, such as a leukemia. A metastatic tumor can arise from a multitude of primary tumor types, including but not limited to those of prostate, colon, lung, breast and liver origin. As used herein, the terms “cancer,” “hyperproliferative,” and “neoplastic” refer to cells having the capacity for autonomous growth, i.e., an abnormal state or condition characterized by rapidly proliferating cell growth. These terms are meant to include all types of cancerous growths or oncogenic processes, metastatic tissues or malignantly transformed cells, tissues, or organs, irrespective of histopathologic type or stage of invasiveness. Proliferative disorders also include hematopoietic neoplastic disorders, including diseases involving hyperplastic/neoplastic cells of hematopoietic origin, e.g., arising from myeloid, lymphoid or erythroid lineages, or precursor cells thereof.

The pharmaceutical compositions of the present invention can also be used to treat a variety of immune disorders, in particular those associated with overexpression or aberrant expression of a gene or expression of a mutant gene. Examples of hematopoietic disorders or diseases include, without limitation, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjogren's syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing, loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, graft-versus-host disease, cases of transplantation, and allergy.

In another embodiment, the invention relates to methods for treating viral diseases, including but not limited to hepatitis C, hepatitis B, herpes simplex virus (HSV), HIV-AIDS, poliovirus, and smallpox virus. Bifunctional molecules containing iRNA therapeutic moieties are prepared as described herein to target expressed sequences of a virus, thus ameliorating viral activity and replication. The bifunctional molecules can be used in the treatment and/or diagnosis of viral infected tissue, both animal and plant. Also, such bifunctional molecules can be used in the treatment of virus-associated carcinoma, such as hepatocellular cancer.

In a further aspect of the invention, the bifunctional compound is prepared conjugated to macromolecular carrier. The macromolecular carrier may be, for example, a liposome which may comprise a polyethylene glycol.

Liposomes in general include vesicles comprising amphiphilic lipids arranged in a spherical layer or bilayers. Liposomes may be unilamellar or multilamellar vesicles. A composition to be delivered is found in the interiors of the vesicles.

A class of liposomes which is useful with nucleic acids is the cationic liposomes, where the lipids are positively charged and are believed to interact with negatively charged DNA molecules to form a stable complex. Certain liposomes that are pH-sensitive or negatively-charged, however, are believed to entrap DNA rather than complex with it. Both cationic and noncationic liposomes have been used to deliver DNA to cells. Information regarding the use of liposomes to deliver nucleic acids is found, for example, in U.S. Published Patent Application No. 2006/62841. Further information regarding the making of liposomes is found, for example, in U.S. Published Patent Application No. 2004/142025.

Liposomes also include sterically stabilized liposomes, which include liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids. The liposome may comprise lipids derivatized with one or more hydrophilic polymers, such as a polyethylene glycol (PEG) moiety. See in this regard, for example, the book Stealth Liposomes (Danilo Lasic & Frank Martin eds., CRC Press 1995).

In a further aspect of the invention, a bifunctional compound is formulated as part of a controlled release formulation in which an additional controlled release mechanism besides the effect of the recruiter moiety is employed to achieve desirable release characteristics. The bifunctional compound is as above, comprising a drug moiety, a linker, and a recruiter moiety.

It is to be understood that while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

### Example 1

### In Vitro Reduction of ApoB Protein Levels

The general method for testing siRNA conjugates is to synthesize the bifunctional compound and test whether the bifunctional version represses apoB protein levels in a cell culture line. ApoB is of clinical interest due to its role in hypercholesterolemia. Initially, genetic databases are searched for candidate siRNAs which are likely to reduce apoB mRNA and protein levels. RT-PCR assays are used to screen lysates of transfected HepG2 liver cells after transfection with the candidate siRNAs. Candidates which reduce the levels of both mRNA transcripts and apoB protein levels are advanced to more detailed screens. These candidates may also be further stabilized against chemical degradation by the use of a phosphorothioate backbone or other modified type of synthetic siRNA. These candidates are then screened in detail for expression in various tissues by an RNA protection assay (RPA). Ideally, dose dependent silencing of apoB expression is observed. Based on the level of expression of apoB vs. amount of siRNAs detected, IC₅₀ values may be calculated. It is desirable to have IC₅₀ values to be less than about 100 nM.

### Example 2

### In Vivo Confirmation of Reduction of ApoB mRNA Levels

Bifunctional siRNAs are introduced via bolus tail vein injection with volumes of about 200 uL on three consecutive days at an siRNA dosage of about 50 mg/kg. 24 hours after the last injection, apoB mRNA levels are assessed using the Northern blot technique: briefly, radiolabelled probes which are complementary to the antisense strands are used to quantify the siRNA in various tissue lysates harvested from the mice as well as mRNA levels. Tissue lysates are run on a gel which is probed with the relevant RNA to detect transcript levels. As a control, animals injected with saline are used. Tissue samples from the liver and jejenum are of particular interest as these are major sights of apoB expression. Levels of apoB protein expression are assessed via conventional Western blot methods.

### Example 3

### Method for Preparing an siRNA-Recruiter Molecule

Various chemistries may be employed to attach the siRNA to the recruiter moiety. One mode of attachment is to use a thiol group at the 5′ end of one of the strands to attach to a recruiter containing an SH moiety. Briefly, siRNAs designed to target an mRNA of interest using standard bioinformatics processes. Typically, for verification of transfection, an mRNA for luciferase may be used or an mRNA for green fluorescent protein (GFP). A typical siRNA for GFP might target coding region 540-565 (relative to the first nucleotide) and would have the sequences: 5′-rArCrUrArCrCrArGrCrArGrAr-ArCrArCrCrCrCTT-3′ and 5′-rGrGrGrGrUrGrUrUrCrUrGrCrUrGrGrUrArGrUTT-3′. This siRNA may then be modified with a thiol group at the 5′ end on the antisense strand. To conjugate to SLF, a modified version of SLF (Holt, et al. supra) is made containing a thiol. Prior to attachment of SLF, the siRNAs are annealed (30 mM HEPES, 2 mM magnesium acetate, 100 mM potassium acetate, pH=7.4) for 1 minute at 90° C. followed immediately by 60 minutes at 37° C. The annealed siRNAs are then desalted using 1% agarose in 100 mM glucose in a 100 μL Eppendorf pipet tip and 100 μL reaction buffer is added (10 mM HEPES, 1 mM ethylenediamine tetraacetic acid, pH 8.0) to adjust to a final siRNA and SLF concentration of 20 μM. The reaction to conjugate SLF to the passenger strand of siRNA takes place for 1 hour at 41° C. Transfection of cells may then be accomplished by incubation with cells. Typical reaction yields for siRNA-SLF are >80% and are verified using HPLC.

### Example 4

### Comparing Cell Transfection Efficiency of siRNA-SLF with siRNA and Lipofectamine

Cell lines are grown according to standard literature methods and are known to one of ordinary skill in the art. Cos-7, C166-GFP, or EOMA-GFP may be used where GFP is green fluorescent protein. The cells are grown in Dulbecco's modified medium (DMEM) supplemented with 10% fetal calf serum (FCS) with added antibiotics. 24 hours after the addition of siRNA and siRNA-SLF, cells are detached from the Petri dish with trypsin and transferred to 24 well plates (300 μL per well). Cells with better transfection efficiencies and better persistence of active siRNA will result in reduced fluorescence of the cell for a longer time period. The siRNAs are added at 25 nM to the transfected cell lines in cell culture media or by transfection with Lipofectamine 2000. Cells containing GFP reporter plasmids are incubated with siRNAs for one week. Gene silencing for cells transfected using siRNA and Lipofectamine and siRNA-SLF and siRNA in buffer is assessed using flow cytometry and Western blot.

For the Western blot, SDS-PAGE (sodium dodecyl sulfate polyacrylamide gel electrophoresis) and immunoblotting along with densitometry can be used to assess the relative expression of GFP in control cells (siRNA with and without Lipofectamine) and test cells transfected with siRNA-SLF.

### Example 5

### Preparation of Thiol Reactive SLF to Conjugate to Thiol-Terminated siRNA

An activated acid derivative of SLF using 1-ethyl-3-(3-dimethylaminopropyl)carbodiimide hydrochloride (EDC) and N-hydroxylsuccinimide (NHS) (10 equivalents EDC to 1 equivalent NHS) is prepared to yield a succinimidyl ester derivative of SLF. The reaction takes place in dimethylformamide (DMF) over the course of two hours at room temperature with a yield of over 82%. This SLF succinimidyl ester is then reacted with ethylene diamine in water and (DMF) and the reaction is initiated by dropwise addition of diisopropylethylamine (DIEA, 400 μL of a 5% solution in DMF). After 30 minutes, the reaction mixture is rotary-evaporated to dryness and the typical yield is 92% to give an SLF-amine derivative. This product is purified by reversed phase HPLC in a two-solvent system with solvent A consisting of water with 0.1% acetic acid and solvent B as a 5/2 mix of ethanol and 1-propanol. Next, the SLF-amine is reacted with succinimidyl-4-(N-maleimidomethyl)cyclohexane-1-carboxylate (SMCC) in a 1:20 ratio in 1.5 mL DMF and 5 μL DIEA and stirred for 50 minutes. The solvent is removed and the dry residue is extracted using 2×20 mL water containing 0.1% acetic acid and 6×20 mL water. Residual dissolved chloroform is expelled by bubbling with nitrogen. Excess water is then removed via lyophilization. Purification takes place by reverse phase HPLC using the same solvent system as described above. The reaction scheme is given in FIG. 4.

### Example 6

### Use of NHS Reactive SLF to Conjugate to Amine-Terminated siRNA to Silence GAPDH Expression and GFP Expression

An NHS ester SLF prepared as in Example 5 is conjugated to the sense strand of RNA corresponding to the glyceraldehyde 3-phosphate dehydrogenase (GAPDH) gene for the purpose of silencing GAPDH. The sense strand is modified as follows where SLF is bound to −5′ GAC UCA UGA CCA CAG UCC A dTdT 3′ and using an unmodified antisense strand as follows: 5′ U GGA CUG UGG UCA UGA GUC dTdT 3′. The RNA is then annealed to form double stranded RNA and administered to a HELA cell culture at a variety of concentrations. GAPDH silencing is then evaluated using a fluorescent indicator to quantitatively detect GAPDH RNA by Quantigene assay (Panomics, Fremont, Calif.). The assay works by binding to GAPDH RNA with oligonucleotides which then bind to a fluorescent indicator. Cell viability is measured using a Cell-Titer Glo Assay (Promega, Madison, Wis.).

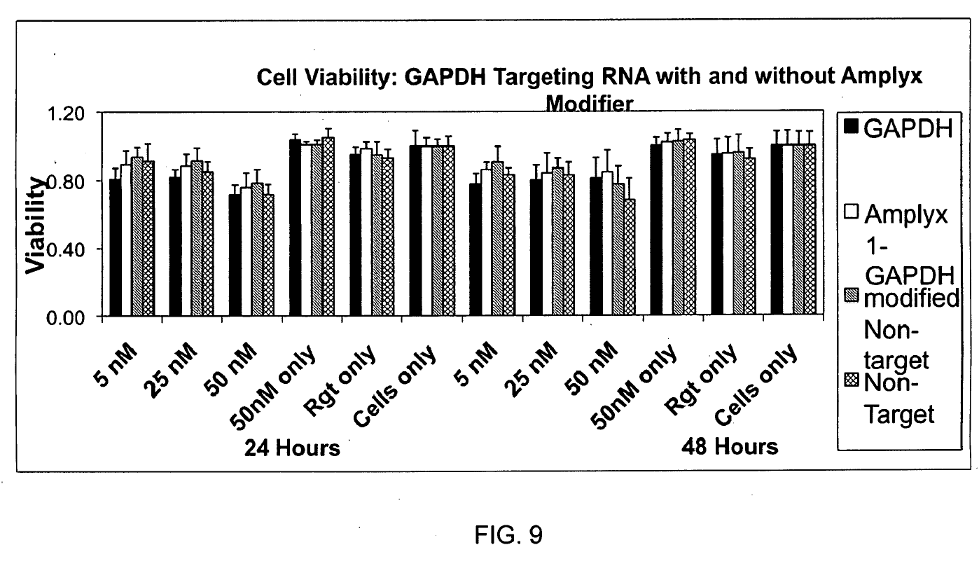

Results for cell viability are shown in FIG. 9. The addition of the SLF modifier appears to improve cell viability when compared with unmodified RNAi. In FIG. 9, “Amplyx-1 GADPH modified” denotes the SLF conjugate prepared as above. The concentrations are noted on the x axis. As a negative control, we employed a non-target RNA not targeting any known protein (a sequence known not to be found in any mammalian cell) and its SLF conjugate. The non-target sequence is sense strand: 5′ CGU ACG CGG AAU ACU UCG AdTdT-3′; antisense strand 1 5′ UCG AAG UAU UCC GCG UAC GdTdT-3′ (a luciferase sequence). The unmodified RNA and conjugate were administered with a cationic liposome except for the 50 nM only samples where aqueous buffer was used. In the figure caption, Rgt only indicates cationic liposome only with no added RNA and Cells only indicates a cell control with no added RNA.

The effect on cell viability is more pronounced at 24 hours than at 48 hours. Non-target controls have similar viability to the GAPDH samples.

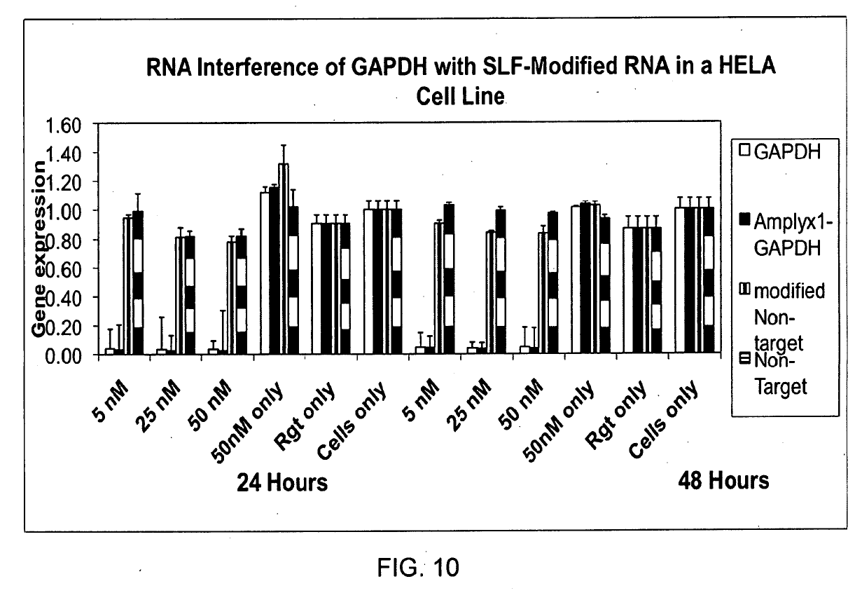

FIG. 10 illustrates that the presence of modifier produces lower knockdown compared to unmodified RNAi. Again, lipofectamine (a cationic liposome) is used to deliver the RNAi into the cell, except for the 50 nM only sample which is in aqueous buffer without lipofectamine, and Rgt only which is lipofectamine with no RNA, and Cells only which have no added reagent. The concentrations are noted on the x axis.

The modified RNA shows about a 25% lower knockdown than unmodified RNA at both 24 and 48 hours. The effect is more pronounced at 24 hours than at 48 hours. Non-target controls have similar negligible effect on GAPDH expression.

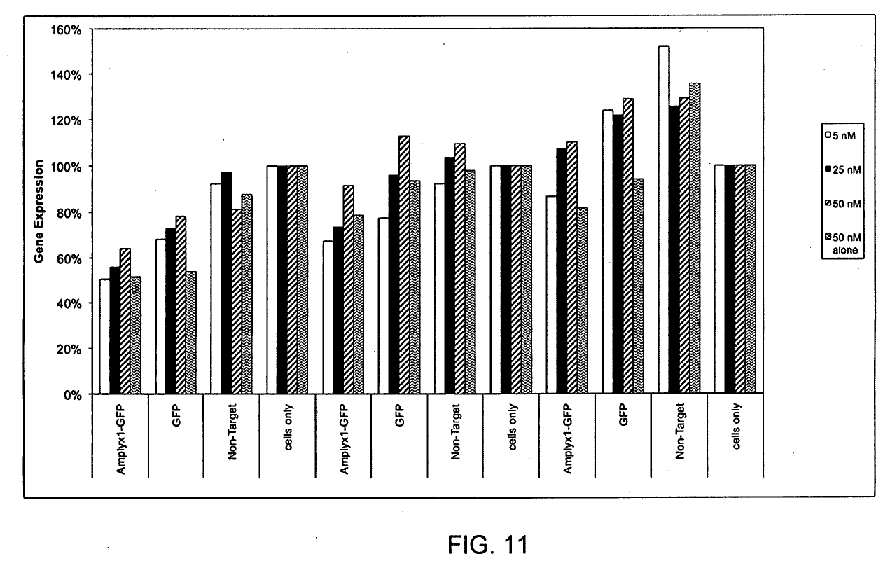

The improved silencing effect achieved by SLF conjugation is further illustrated by an experiment where SLF is attached to the sense RNA coding for the green fluorescent protein (GFP). FIG. 11 illustrates improved knockdown of green fluorescent protein expression as measured by GFP fluorescence with SLF modified RNA compared to unmodified RNA. The non-target control showed no significant reduction in GFP expression.

The sequences for GFP used were: sense strand: 5′ ArCrUrArCrCrArGrCrArGrArArCrArCrCrCrCTT-3′; antisense strand: 3′-TTUGrArUrGrGrUrCrGrUrCrUrUrGrUrGrGrGrGr-5′ where the sense strand is modified with SLF. In the figure, Amplyx1-GFP indicates SLF modified RNA, GFP is unmodified. Non-target denotes an irrelevant sequence (sense strand: 5′ CGU ACG CGG AAU ACU UCG AdTdT-3′; antisense strand 1 5′ UCG AAG UAU UCC GCG UAC GdTdT-3′). Cells only indicates a control with cells only, and concentrations are in nM as indicated. 50 nM alone means the RNA was added to the cells with no cationic liposome to assist with intracellular delivery. The cell line employed in this experiment was C166-GFP.

We claim:

1. A method for improving at least one pharmacokinetic property and efficacy of an RNAi therapeutic moiety upon administration to a host, the method comprising:
administering to the host an effective amount of a bifunctional compound comprising the RNAi therapeutic or an active derivative, fragment or analog thereof and a recruiter moiety,
wherein the recruiter moiety is less than 1200 daltons and is a non-immunosuppressive derivative, fragment, or analog of a peptidyl prolyl isomerase binding molecule and
wherein the bifunctional compound has at least one modulated pharmacokinetic property upon administration to the host as compared to the RNAi therapeutic moiety.

2. The method according to claim 1, wherein the pharmacokinetic property is selected from the group consisting of half-life, hepatic first-pass metabolism, volume of distribution, and degree of blood protein binding.

3. The method according to claim 1, wherein the intracellular distribution of the bifunctional is increased by at least 10% relative to the RNAi therapeutic.

4. The method according to claim 1, wherein the intracellular distribution of the bifunctional is increased by at least 20% relative to the RNAi therapeutic.

5. The method according to claim 1, wherein the bifunctional compound is administered in a pharmaceutical preparation.

6. The method according to claim 1, wherein the host is a mammal.

7. The method according to claim 1 where the recruiter moiety has a mass of less than 1100 daltons.

8. The method according to claim 1 where there is a covalent linker between the RNAi therapeutic moiety and the recruiter moiety.

9. The method according to claim 1 wherein at least two bifunctional moieties containing at least two different RNAi therapeutic moieties are administered to a host.

10. The method according to claim 1 wherein a bifunctional moiety containing at least two different RNAi therapeutic moieties is administered to a host.

11. The method according to claim 1 where the RNAi therapeutic moiety comprises an RNA modified to include at least one of: a phosphothioate, a boranophosphonate, 2′-O-methyl RNA, 2′-deoxy-2′-fluoro RNA, or a locked nucleic acid.

12. The method according to claim 1 where the RNAi therapeutic moiety contains RNA duplexes of at least 18 nucleotides in length.

13. The method according to claim 1 where the RNAi therapeutic moiety contains RNA duplexes of at least 21 nucleotides in length.

14. The method according to claim 1, wherein the RNAi therapeutic moiety comprises an RNA duplex of at least 27 nucleotides in length.

15. The method of claim 1 wherein the bifunctional compound has improved accumulation in the brain compared with the RNAi therapeutic moiety.

16. The method according to claim 1 where the RNAi therapeutic moiety contains asymmetrical siRNA's with 5′ blunt ends and two-nucleotide overhangs at the 3′ ends.

17. The method according to claim 1 where the RNAi therapeutic moiety contains adenine or uracil at the 5′ end of the antisense strand.

18. The method according to claim 1 where the RNAi therapeutic moiety does not contain known sites for mRNA binding in the 5′ or 3′ untranslated region (UTR).

19. The method according to claim 1 where the recruiter moiety is bound to the passenger (antisense) strand of the RNAi therapeutic moiety.

20. The method according to claim 1 where the recruiter moiety is bound to the guide (sense) strand of the RNAi therapeutic moiety.

21. A method for improving at least one pharmacokinetic property and efficacy of an RNAi therapeutic moiety upon administration to a host, the method comprising:
administering to the host an effective amount of a bifunctional compound comprising the RNAi therapeutic or an active derivative, fragment or analog thereof and a recruiter moiety,
wherein the recruiter modulating moiety binds to at least one intracellular protein and
wherein the bifunctional compound has at least one modulated pharmacokinetic property upon administration to the host as compared to the RNAi therapeutic moiety and the bifunctional compound is prepared conjugated to macromolecular carrier.

22. The method of claim 21 where the carrier is a liposome containing polyethylene glycol moieties.

23. A composition for modulating the level of an mRNA, comprising:
(a) a RNAi therapeutic moiety or an active derivative, fragment or analog thereof, and
(b) a recruiter moiety, wherein the recruiter moiety is adapted to bind to at least one substantially non-membrane bound intracellular protein.

24. The composition of claim 23, wherein the RNAi therapeutic moiety comprises a member selected from the group consisting of shRNA, miRNA, and siRNA.

25. The composition of claim 24, wherein the RNA therapeutic comprises an siRNA containing a 5′ end and a 3′ end, and wherein the 5′ end of the siRNA comprises a blunt end and the 3′ end of the siRNA comprises a two-nucleotide overhang.

26. The composition of claim 24, wherein the RNA therapeutic comprises an siRNA and the siRNA comprises an RNA duplex comprising a sense and an antisense strand.

28. The composition of claim 26, wherein one or more of the 5′ ends and 3′ ends of the sense and/or antisense strands comprise an untranslated region (UTR).

29. The composition of claim 28, wherein none of the 5′ ends and 3′ ends comprise a site for mRNA binding in the untranslated region (UTR).

30. The composition of claim 27, wherein the RNAi therapeutic moiety has a molecular weight in the range of about 4,000 daltons to about 50,000 daltons.

31. The composition of claim 23, the RNAi therapeutic moiety comprises RNA modified to include at least one of: a phosphothioate; a boranophosphonate; 2′-O-methyl RNA; 2′-deoxy-2′-fluoro RNA; and a locked nucleic acid.

32. The composition of claim 23, wherein the RNAi therapeutic moiety contains RNA duplexes.

33. The composition of claim 32, wherein the RNA duplexes are at least 18 nucleotides in length.

34. The composition of claim 23, wherein the recruiter moiety has a molecular weight from about 500 daltons to about 2000 daltons.

35. The composition according to claim 23 where the substantially non-membrane bound intracellular protein comprises a protein selected from the group consisting of: FK506 binding proteins, cyclophilin, tubulin, actin, heat shock proteins, and peptidyl prolyl isomerases.

36. The composition according to claim 35, wherein the recruiter moiety binding to the recruited target is adapted to sterically hinder the ability of a metabolic enzyme to degrade the siRNA therapeutic moiety when the recruiter molecule is bound to the protein.

37. The composition according to claim 36, wherein the enzyme comprises an RNAse enzyme.

38. The composition of claim 26, wherein the recruiter moiety is bound to the antisense (passenger) strand of the siRNA therapeutic moiety.

39. The composition of claim 23, further comprising a linking group between the RNAi therapeutic moiety and the recruiter.

40. The composition of claim 23, wherein the linking group comprises a covalent linker.

41. The composition of claim 23, further comprising at least two RNAi therapeutic moieties and at least two recruiter moieties.

42. The composition of claim 23, further comprising a pharmaceutically acceptable carrier.

43. The composition of claim 42, wherein the pharmaceutically acceptable carrier comprises a liposome.

44. The composition of claim 43, wherein the liposome comprises a polyethylene glycol moiety.

45. The composition of claim 42, wherein said composition is formulated in the form of a tablet, capsule, and a parenteral formulation.

46. The composition of claim 43, wherein said composition comprises a sustained release formulation.

47. A method for improving at least one pharmacokinetic property and efficacy of an RNAi therapeutic moiety upon administration to a host, the method comprising:
administering to the host an effective amount of a bifunctional compound comprising the RNAi therapeutic or an active derivative, fragment or analog thereof and a recruiter moiety,
wherein the recruiter moiety is less than 1200 daltons and binds to an intracellular protein and
wherein the RNAi therapeutic is used to accomplish exon skipping.

48. The method according to claim 47, wherein the intracellular distribution of the bifunctional is increased by at least 10% relative to the RNAi therapeutic.

49. The method according to claim 47, wherein the intracellular distribution of the bifunctional is increased by at least 40% relative to the RNAi therapeutic.

50. The method according to claim 47 where the recruiter moiety has a mass of less than 1200 Daltons and binds to a peptidyl prolyl isomerase.

51. The method according to claim 47 where there is a covalent linker between the RNAi therapeutic moiety and recruiter.

52. The method according to claim 1 where at least two bifunctional moieties containing at least two different RNAi therapeutic moieties are administered to a host.

53. The method according to claim 47 where the RNAi therapeutic moiety contains at least one of the following types of modified RNA molecules: phosphothioate, boranophosphonate, 2′-O-methyl RNA, 2′-deoxy-2′-fluoro RNA, or a locked nucleic acid.

54. The method according to claim 47 where the RNAi therapeutic moiety contains at least about 30% G/C content, at least 40% G/C content, or at least about 50% G/C content.

55. The method according to claim 47 where the RNAi therapeutic moiety does not contain known sites for mRNA binding in the 5′ or 3′ untranslated region (UTR).

56. A method of treating or preventing a disease condition characterized by expression of a gene, comprising the steps of administering to a patient a bifunctional compound comprising a therapeutic moiety which acts on the RNAi mechanism in such a way as to affect the expression of the gene and a recruiter moiety, wherein the recruiter moiety has a molecular weight of less than 1200 daltons and binds to at least one substantially non-membrane bound intracellular protein.

57. The method of claim 56, wherein the uptake of the bifunctional molecule in the patient's cells is not receptor-mediated.

58. The method of claim 56, wherein the disease condition is muscular dystrophy, macular degeneration, leukemia, or cystic fibrosis.

59. The method of claim 56, wherein the recruiter moiety is not a lipid or folate.

60. The method of claim 56, wherein the recruiter moiety does not target any cell-surface receptor when the bifunctional molecule is in extracellular space.

61. The method of claim 56, wherein the bifunctional compound reduces gene expression to a greater extent than the unmodified therapeutic moiety.

62. The method of claim 56, wherein the bifunctional compound is administered without a liposome.

63. The method of claim 56, wherein the bifunctional compound improves cell viability compared to the unmodified therapeutic moiety.


SEQUENCE LISTING (10 sequences)

SEQ ID NO: 1 - DNA, 21 bp, Artificial Sequence
  Combined DNA/RNA molecule; synthetic RNAi oligonucleotide
acuaccagcagaacacccctt

SEQ ID NO: 2 - DNA, 21 bp, Artificial Sequence
  Combined DNA/RNA molecule; synthetic RNAi oligonucleotide
gggguguucugcugguagutt

SEQ ID NO: 3 - DNA, 21 bp, Artificial Sequence
  Combined DNA/RNA Molecule; synthetic RNAi oligonucleotide
gacucaugaccacaguccatt

SEQ ID NO: 4 - DNA, 21 bp, Artificial Sequence
  Combined DNA/RNA Molecule Synthetic RNAi oligonucleotide
uggacuguggucaugaguctt

SEQ ID NO: 5 - DNA, 21 bp, Artificial Sequence
  Combined DNA/RNA Molecule Synthetic RNAi oligonucleotide
cguacgcggaauacuucgatt

SEQ ID NO: 6 - DNA, 21 bp, Artificial Sequence
  Combined DNA/RNA Molecule Synthetic RNAi oligonucleotide
ucgaaguauuccgcguacgtt

SEQ ID NO: 7 - DNA, 21 bp, Artificial Sequence
  Combined DNA/RNA Molecule Synthetic RNAi oligonucleotide
acuaccagcagaacacccctt

SEQ ID NO: 8 - DNA, 21 bp, Artificial Sequence
  Combined DNA/RNA Molecule Synthetic RNAi oligo
ttugauggucgucuugugggg

SEQ ID NO: 9 - DNA, 21 bp, Artificial Sequence
  Combined DNA/RNA Molecule Synthetic RNAi oligonucleotide
cguacgcggaauacuucgatt

SEQ ID NO: 10 - DNA, 21 bp, Artificial Sequence
  Combined DNA/RNA Molecule Synthetic RNAi oligonucleotide
ucgaaguauuccgcguacgtt